(19) United States
(12) Patent Application Publication
(10) Pub. No.: US 2019/0015576 A1
(43) Pub. Date: Jan. 17, 2019

(54) MEDICAL INJECTION AND SUCTION DEVICE

(71) Applicant: Benepet Co., Ltd., Taipei City (TW)

(72) Inventor: Ta-Lun Tan, Taipei City (TW)

(73) Assignee: Benepet Co., Ltd., Taipei City (TW)

(21) Appl. No.: 15/877,770

(22) Filed: Jan. 23, 2018

(30) Foreign Application Priority Data
Jul. 11, 2017 (TW) 106210195

(51) Int. Cl. A61M 1/28; A61M 5/145; A61M 1/00

(52) U.S. Cl. CPC A61M 1/285; A61M 5/14546; A61M 1/0084; A61M 2205/3334; A61M 2205/3337; A61M 2205/6072; A61M 2205/3561; A61M 2205/3306; A61M 2205/505; A61M 2205/18

(57) ABSTRACT

A medical injection and suction device includes: a housing that encloses a driving motor; a syringe mount disposed on a wall of an upper side of the housing; a plurality of valves disposed on the wall of the upper side of the housing and on a side of the syringe mount; a slider that moves toward or away from the housing and is disposed on a lateral side of the housing; a driving rod connected to the slider; and at least one slide rod. The driving rod extends through a wall of the lateral side of the housing. An end of the driving rod is connected to the driving motor. The driving motor drives the driving rod to move the slider. An end of the at least one slide rod is connected to the wall of the lateral side of the housing. The slider is disposed in a slidable manner on the slide rod.

### TECHNICAL FIELD

The invention relates to a medical injection and suction device, in particular, to a medical injection and suction device capable of automatically and stably and precisely injecting and drawing medical infusions or waste fluids.

### BACKGROUND

In the medical field, medical personnel often have to manually perform an injection or suction procedure in a steady way when injecting medical infusions (drips, blood, or dialysate) or suctioning waste fluids (lung water, ascites, pleural effusion, pericardial effusion, or waste dialysate from dialysis) from the body. When performed manually, however, the injection of medical infusions or the suction of waste fluids is neither precise nor performed at a steady rate. When the patient is physically sensitive (for example, when the patient is a small animal), the amount of injection or suction must be very precise, and the rate of injection or suction must be very slow and steady. Otherwise, the procedure is likely to cause harm to the patient. Thus, not only is the manual performance of the procedure dangerous but also requires heavy manpower.

### SUMMARY

One or more embodiments disclosed herein provide a medical injection and suction device that: (1) can precisely quantify the injection amount of medical infusions and suction amount of waste fluids; (2) can inject medical infusions in a slow but continuous and stable manner, (3) has the function of preventing catheter backflow or blockage, and can automatically inject infusions without being manually performed or monitored by a medical personnel, which is able to save a lot of manpower; (4) has a stable and safe structure and prolonged product life; and (5) has the function of preventing cross-infection resulting from repeated use of consumables used in conjunction therewith.

According to one aspect, one or more embodiments disclosed herein relate to a medical injection and suction device, comprising: a housing that encloses a driving motor; a syringe mount disposed on a wall of an upper side of the housing; a plurality of valves disposed on the wall of the upper side of the housing and on a side of the syringe mount; a slider that moves toward or away from the housing and is disposed on a lateral side of the housing; a driving rod connected to the slider, wherein the driving rod extends through a wall of the lateral side of the housing, an end of the driving rod is connected to the driving motor, and the driving motor drives the driving rod to move the slider, and at least one slide rod, wherein an end of the at least one slide rod is connected to the wall of the lateral side of the housing, and the slider is disposed in a slidable manner on the slide rod.

In one aspect, the syringe mount fixes a syringe to the housing. The plurality of valves close and open a plurality of catheters connected to the syringe. The slider is connected to a plunger of the syringe and pushes and pulls the plunger of the syringe to inject and suction liquids.

In one aspect, the slider is not disposed on the upper side of the housing.

In one aspect, the medical injection and suction device further comprises a stop block that stops the slider from moving off the slide rod and is disposed at an end of the slide rod.

In one aspect, the medical injection and suction device further comprises a control device that is electrically connected to the driving motor and controls the driving of the driving motor to move the slider. The control device is electrically connected to the plurality of valves and controls the closing and opening of the valves.

In one aspect, the medical injection and suction device further comprises a flow sensor that senses a flow velocity and a flow direction of a liquid in a catheter.

In one aspect, the control device is electrically connected to the flow sensor and receives a signal of the flow velocity and the flow direction sensed by the flow sensor. The control device stops the movement of the slider and issues an alarm in response to the flow velocity being lower than a predetermined value, or the control device stops the movement of the slider and issues an alarm in response to the flow direction not being a predetermined direction.

In one aspect, the medical injection and suction device further comprises a displacement sensing device including a resistance ruler. The displacement sensing device measures a displacement of the slider by reading a difference of resistance value of the resistance ruler.

In one aspect, the control device is electrically connected to the displacement sensing device and receives a signal of the displacement of the slider measured by the displacement sensing device. The control device stops the movement of the slider in response to the displacement of the slider measured by the displacement sensing device reaching a predetermined displacement to execute a predetermined injection amount or a predetermined suction amount.

In one aspect, the control device comprises a timing unit that calculates and measures time. The control device drives the driving motor for a predetermined amount of time for injection or a predetermined amount of time for suction to complete the predetermined displacement of the slider to execute a predetermined injection rate or a predetermined suction rate.

In one aspect, the medical injection and suction device further comprises a barcode scanner electrically connected to the control device. The barcode scanner sends a start signal to the control device in response to the completion of scanning of a correct barcode, and the control device cannot be started until the start signal is received.

In one aspect, the medical injection and suction device further comprises a control device. The control device comprises an input interface for inputting a patient data, an output interface for outputting the patient data, and a memory that stores the patient data.

In one aspect, the medical injection and suction device further comprises a control device comprising a transceiving unit that uploads the patient data or an alarm in the control unit to a cloud terminal, a remote computer, or a mobile device.

One or more embodiments disclosed herein are configured to achieve the following:

(1) The plurality of valves close and open a plurality of catheters connected to the syringe. The slider is connected to a plunger of the syringe and pushes and pulls the plunger of the syringe to inject and suction liquids. During a suction procedure, a first valve is opened to keep a first catheter conductive. The slider moves a specific distance away from the housing and pulls the plunger of the syringe to draw a liquid (infusion or waste fluid) into the syringe. The displacement sensing device senses the displacement of the slider to precisely quantify the injection amount of the medical infusion and the suction amount of the waste fluid, and the displacement sensing device measures the displacement of the slider through a difference of resistance value of the resistance ruler rather than by using a conventional grating, which increases the precision of quantification. During an injection procedure, the first valve is closed to close the first catheter and a second valve is opened to keep a second catheter conductive. The slider moves a specific distance toward the housing and pushes the plunger of the syringe to inject or expel a fluid (infusion or waste fluid).

(2) The medical injection and suction device of one or more embodiments disclosed herein provide the slow but steady injection of a liquid of the syringe into a patient's body at a specific rate by applying a steady force to the plunger of the syringe in order to overcome the instability during injection or suction resulting from pushing the fluid in the catheter traditionally by manually pushing the plunger of the syringe or by gravity.

(3) In the medical injection and suction device of one or more embodiments disclosed herein, when the flow sensor detects that the flow velocity in the catheter is too low or the flow is not in the predetermined direction, the control device stops moving to push or pull the syringe plunger and sends an alarm to a medical person. Therefore, the medical injection and suction device prevents catheter backflow or blockage and does not require the personal operation and monitoring of medical personnel, thereby saving a lot of manpower.

(4) In the medical injection and suction device of one or more embodiments disclosed herein, the slider is disposed on a lateral side of the housing to move toward or away from the housing instead of being provided on an upper side of the housing to move on the wall of the upper side of the housing. Therefore, the rod (for example, a driving rod) connecting the slider to the driving motor does not extend through the wall of the upper side of the housing to form a hole or slit through the wall of the upper side. Instead, it extends through the wall of a lateral side of the housing so that liquid does not flow into the housing through a hole or slit of the wall of the upper side when the liquid leaks from the syringe, damaging the medical injection and suction device. By this stable and safe device structure the life of the product is prolonged.

(5) In the medical injection and suction device of one or more embodiments disclosed herein, the consumables used in conjunction with the device have a barcode on the package, and the control device can be started into operation for a specific amount of time only after the barcode scanner scans the barcode on the package. The barcode cannot be used to start the control device again after being scanned so as to ensure that each time the medical injection and suction device is used, new, clean consumables are used in conjunction with the device to avoid the repeated use of consumables which leads to cross-infection.

### BRIEF DESCRIPTION OF DRAWINGS

FIG. 1 is a schematic diagram of the external appearance of the medical injection and suction device according to one or more embodiments.

FIG. 2 is a schematic diagram of the internal structures of the medical injection and suction device according to one or more embodiments.

FIG. 3 is a schematic diagram of the external appearance of a peritoneal dialysis catheter for animals which can be used in conjunction with the medical injection and suction device according to one or more embodiments.

FIG. 4 is a flow chart of the use of the peritoneal dialysis device for animals in conjunction with the peritoneal dialysis catheter for animals according to one or more embodiments.

### REFERENCE NUMERALS

100 Medical injection and suction device
110 Housing
111 Driving motor
112 Wall of the upper side
113 Wall of the lateral side
120 Syringe mount
121 Fixing member
131 First valve
132 Second valve
133 Third valve
140 Slider
150 Driving rod
160 Slide rod
161 Stop block
170 Control device
180 Flow sensor
190 Displacement sensor
191 Resistance ruler
192 Guiding rod
193 Probe module
200 Barcode scanner
1 Peritoneal dialysis catheter for animals
10 Dialysate catheter
20 Injection catheter
24 Peritoneal catheter
30 Waste fluid catheter
33 Waste fluid container
40 Syringe catheter
41 Syringe
42 Plunger
50 Four-way connector

### DETAILED DESCRIPTION

One or more embodiments disclosed herein will be described in detail with reference to the attached drawings. The following descriptions of one or more embodiments are intended for exemplary purposes only, and should not be used to limit the one or more embodiments disclosed herein.

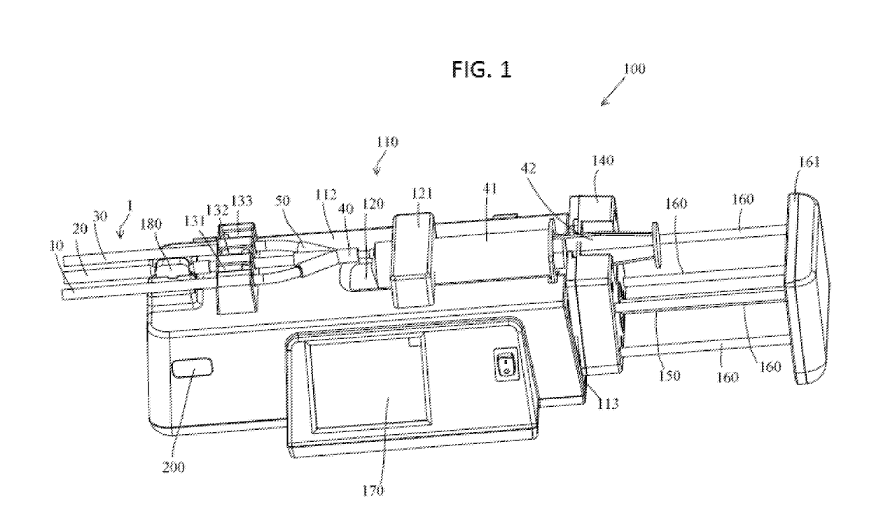

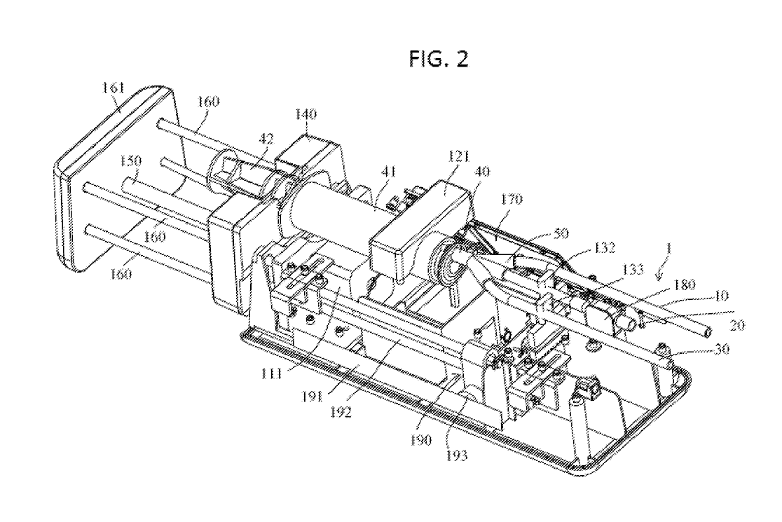

Referring now to FIG. 1 and FIG. 2, FIG. 1 is a schematic diagram of the external appearance of the medical injection and suction device according to one or more embodiments, and FIG. 2 is a schematic diagram of the internal structures of the medical injection and suction device according to one or more embodiments. In one or more embodiments, a medical injection and suction device 100 includes a housing 110 that encloses a driving motor 111; a syringe mount 120 disposed on a wall 112 of an upper side of the housing 110; a plurality of valves 131, 132, 133 disposed on the wall 112 of the upper side of the housing 110 and on a side of the syringe mount 120; a slider 140 that moves toward or away from the housing 110 and is disposed on a lateral side of the housing; a driving rod 150 connected to the slider 140, wherein the driving rod 150 extends through a wall 113 of the lateral side of the housing 110, an end of the driving rod 150 is connected to the driving motor 111, and the driving motor 111 drives the driving rod 150 to move the slider 140; at least one slide rod 160, wherein an end of the at least one slide rod is connected to the wall 113 of the lateral side of the housing 110, and the slider 140 is disposed in a slidable manner on the slide rod 160; a control device 170 electrically connected to the driving motor 11 and controls the driving of the driving motor 111 to move the slider 140, wherein the control device 170 is electrically connected to the plurality of valves 131, 132, 133 and controls the closing and opening of the valves; a flow sensor 180 that senses a flow velocity and a flow direction of a liquid in a catheter; and a displacement sensing device 190 that senses a displacement of the slider 140.

The housing 110 includes a plurality of mechanical components and a plurality of electronic components such as the driving motor 111, a portion of the driving rod 150, the control device 170, the flow sensor 180, the displacement sensor 190, and the like. The syringe mount 120 fixes a syringe 41 to the wall 112 of the upper side of the housing 110. In one or more embodiments, a fixing member 121 may also be provided on the syringe mount 120, as shown in FIG. 1. A spring is provided at the base of the fixing member 121 so that the fixing member 121 clamps and fixes the syringe 41 to the wall 112 of the upper side of the housing 110. This configuration of the fixing member 121 is only an example of its implementation. Alternatively, the fixing member 121 may also be implemented as other components such as a snap-fit element, a magnetic fastener or the like to fix the syringe.

In one or more embodiments, a medical injection and suction device 100 includes a plurality of valves 131, 132, 133. The number of the valves can be two, three or even more. For example, in one or more embodiments shown in FIG. 1 and FIG. 2, the number of the valves is three. The plurality of valves 131, 132, 133 are disposed on the wall 112 of the upper side of the housing 110 and are located on the front-end side (the opposite side to the position of the plunger) of the syringe mount 120 and are configured to close (shut) and open (make conductive) a plurality of catheters 10, 20, 30 connected to the syringe. The plurality of catheters 10, 20, 30 respectively can be used for suctioning or injecting infusions and for expelling or suctioning waste fluids. The plurality of valves 131, 132, 133 may be implemented as solenoid valves. The opening or closing of the valves may be controlled by the magnetic force generated by energizing/de-energizing the solenoids of the solenoid valves. Therefore, an automatic control of the valves may be realized by electrically connecting the control device 170 to the plurality of valves.

In one or more embodiments, the driving rod 150 is a threaded rod and is connected to the slider 140. The slider 140 rotatably engages the threads on the driving rod 150 through the hole in the slider 140. Further, the driving rod 150 extends through the wall 113 of the lateral side of the housing 110. An end of the driving rod 150 is drivingly connected to the driving motor 111, and the driving motor 111 rotationally drives the driving rod 150 to move the slider 140.

The number of the slide rod 160 is at least one. Alternatively, there may be more than one slide rod 160. For example, in FIG. 1 and FIG. 2, the number of the slide rod 160 is four, passing through the four corners of the slider 140. An end of the slide rod is connected to the wall 113 of the lateral side of the housing 110, and the slider 140 is disposed in a slidable manner on the slide rod 160 to prevent the slider 140 from rotating in the circumferential direction along with the driving rod when the driving rod rotates. In this way, the slider 140 will only move in the direction in which the threads of the driving rod advances (i.e., the direction of the driving rod or the slide rod). As shown in FIG. 1 and FIG. 2, the medical injection and suction device further comprises a stop block 161 disposed at an end of the slide rod 160 and that stops the slider 140 from moving off the slide rod.

In one or more embodiments, as shown in FIG. 1 and FIG. 2, the slider 140 is provided with a groove that engages or fixes the plunger 42 of the syringe 41. When the driving motor 111 drives or moves the slider 140, the slider 140 pushes and pulls the plunger of the syringe to inject the liquid in the barrel of the syringe or to draw liquid (such as infusion or waste fluid) into the barrel of the syringe.

A technical feature of the medical injection and suction device according to one or more embodiments is that the slider 140 is disposed on a lateral side of the housing 110 to move toward or away from the housing 110 instead of being provided on an upper side of the housing 110 to move on a wall 112 of the upper side of the housing. Therefore, the rod (for example, a driving rod) connecting the slider 140 to the driving motor 111 does not extend through the wall 112 of the upper side of the housing to form a hole or slit through the wall 112 of the upper side. Instead, it extends through a wall 113 of a lateral side of the housing. Therefore, another technical feature of the medical injection and suction device according to one or more embodiments is that the wall 112 of the upper side of the housing does not have openings so that liquid does not flow into the housing 110 through a hole or slit of the wall 112 of the upper side when the liquid leaks from the syringe, damaging the medical injection and suction device. By this stable and safe device structure the life of the product is prolonged.

In one or more embodiments, the control device 170 includes a central processing unit (CPU), a memory, a timer, an input interface, and an output interface. The control device 170 is implemented as a microcomputer having a microprocessor as the central processing unit (CPU). A plurality of functions of the central processing unit are integrated into a single chip integrated circuit (IC). The control device 170 may also be implemented as a microcontroller, also referred to as a single-chip microcomputer, which is a type of microcomputer in which a central processing unit, a memory, a timer, and a variety of input and output interfaces are integrated in an integrated circuit chip. The memory is used to store instructions, data, and patient information, including volatile memory such as Dynamic Random Access Memory (DRAM) or Static Random Access Memory (SRAM), as well as non-volatile memory such as Read Only Memory (ROM), flash memory, solid state drive, magnetic drive, and the like. In one or more embodiments, the input interface and the output interface are a touch-control liquid crystal screen for inputting a patient data and outputting the patient data such as patient name, medical record number, weight, total infusion volume, infusion volume per kg body weight, amount of time for injection, injection rate, amount of time for suction, suction rate, residence time and so on. In one or more embodiments, the control device 170 further includes a transceiving unit that uploads the patient data in the control unit to a cloud terminal, a remote computer or a mobile device, or sends an alarm to a cloud terminal, a remote computer, or a mobile device to alert medical personnel. In addition, in one or more embodiments, data from the cloud terminal, the remote computer, or the mobile device may be received by the transceiving unit, for example, to carry out online data update or software update.

The control device 170 is electrically connected to the driving motor 111, the plurality of valves 131, 132, 133, the flow sensor 180, and a displacement sensor 190. The control device 170 controls the driving of the driving motor 111 to move the slider 140 for injection or suction. The control device 170 controls the closing and opening of the valves 131, 132, 133 to select the catheters 10, 20, 30 for injection or suction. The control device 170 receives a signal of the flow velocity and the flow direction measured by the flow sensor 180 to monitor the status of the fluid in the injection catheter 20 in real time. The control device 170 receives a signal of the displacement of the slider 140 measured by the displacement sensing device 190 to measure a displacement of the slider 140 and calculates the amount of injected liquid and the amount of suctioned liquid based on the displacement of the slider.

In one or more embodiments, the flow sensor 180 includes a light sensing unit. The fluid in the injection catheter 20 flows through a rotating member and drives the rotating member to rotate. The rotating member rotates to change the frequency of the light received by the light sensing unit, and the rotating speed and the rotating direction of the rotating member are calculated to deduce the flow velocity and the flow direction of the fluid. For a specific structure of the flow sensor 180, reference may be made to the prior application of Taiwan Patent Application No. 106113057 by the present inventor. When the flow velocity is lower than a predetermined value, it indicates that the catheter 20 may be blocked, and the control device 170 stops the movement of the slider 140, immediately interrupting the injection or suction procedure and sends an alarm via a transceiving device 200 to a cloud terminal, a remote computer, or a mobile device such as a mobile phone of a medical person. Alternatively, when the flow direction is not a predetermined direction (for example, the flow direction detected is the flow direction of suction while it should have been the flow direction of injection), it indicates that the catheter may be severely blocked, and the control device 170 stops the movement of the slider 140, immediately interrupting the injection or suction procedure and sends an alarm via the transceiving device to a cloud terminal, a remote computer, or a mobile device such as a mobile phone of a medical person.

In one or more embodiments, the displacement sensing device 190 includes a probe module 193, a guiding rod 192, and a resistance ruler 191. The displacement sensing device 190 is disposed inside the housing 110. The guiding rod 192 extends through the wall 113 of the lateral side of the housing 110. One end of the guiding rod 192 is connected to the slider 140, and the other end of the guiding rod 192 is connected to the probe module 193. The resistance ruler 191 is disposed on a side of the probe module 193. The guiding rod 192 pushes the probe module 193 to move along the direction of the resistance ruler 191. The probe module 193 contacts the resistance ruler 191 to measure a displacement of the slider 140 by reading a change of resistance value of the resistance ruler 191. In one or more embodiments, measuring the displacement of the slider 140 by using the resistance ruler 191 is more precise than measuring the displacement of the slider 140 by using a grating as in the prior art. For a specific structure of the displacement sensing device 190, reference may be made to Taiwan Patent Application No. 106207198 previously filed by the present inventor.

A predetermined injection amount or a predetermined suction amount is input to the control device 170 through the input interface. The control device 170 calculates a predetermined displacement of the slider 140 (for example, a forward distance or a backward distance) based on the predetermined injection amount or suction amount. The control device 170 drives the driving motor 111i to move the slider 140. When the displacement of the slider sensed by the displacement sensing device reaches the predetermined displacement, the control device 170 stops the movement of the slider to execute the predetermined injection amount or the predetermined suction amount.

A predetermined injection rate or a predetermined suction rate is input to the control device 170 through the input interface. The control device 170 calculates a predetermined amount of time for injection or a predetermined amount of time for suction based on the predetermined injection rate or the predetermined suction rate. The timing unit is used to calculate and measure time. The control device drives the driving motor for the predetermined amount of time for injection or the predetermined amount of time for suction to complete the predetermined displacement of the slider to execute the predetermined injection rate or the predetermined suction rate.

In one or more embodiments, the medical injection and suction device 100 further comprises a barcode scanner 200 electrically connected to the control device 170. The consumables used in conjunction with the medical injection and suction device 100 have a barcode on the package. Upon scanning the barcode on the package, the barcode scanner 200 sends a start signal to the control device 170. The control device 170 can be started for a specific amount of time (for example, a predetermined amount of time for one treatment) only after receiving the start signal. The barcode cannot be used again to start the control device 170 after being scanned so as to ensure that each time the medical injection and suction device 100 is used, new, clean consumables must be used to avoid repeated use of the consumables which leads to cross-infection.

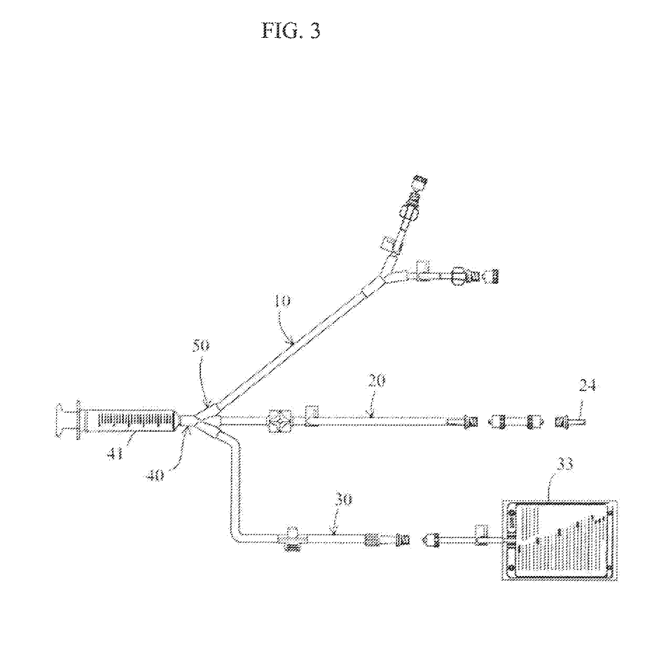

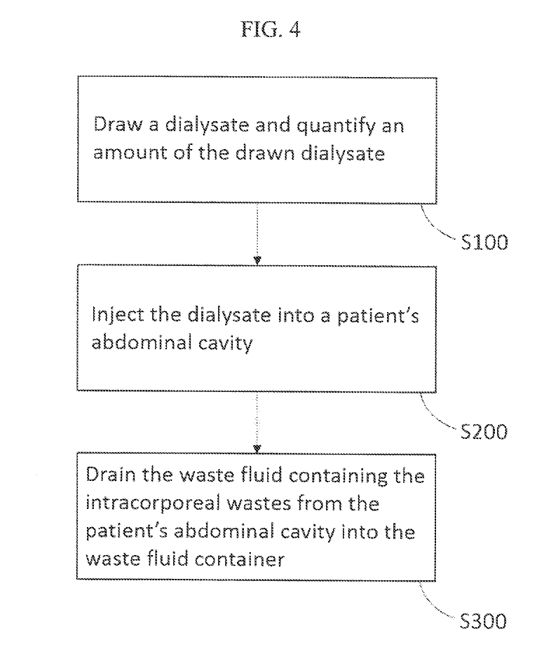

Since the medical injection and suction device 100 according to one or more embodiments disclosed herein can be used to perform peritoneal dialysis, a method of using the medical injection and suction device 100 in conjunction with a peritoneal dialysis catheter 1 for animals is provided below. Referring to FIG. 3 and FIG. 4, FIG. 3 is a schematic diagram of the external appearance of a peritoneal dialysis catheter 1 for animals. The peritoneal dialysis catheter 1 for animals is used as a consumable in conjunction with the medical injection and suction device 100. FIG. 4 is a flow chart of the use of the peritoneal dialysis device 100 for animals in conjunction with the peritoneal dialysis catheter 1 for animals according to one or more embodiments.

Step S100: A dialysate is drawn and an amount of the dialysate to be injected is quantified. The medical injection and suction device 100 opens the first valve 131 to make the dialysate catheter 10 conductive and close the second valve 132 and the third valve 133 to close the injection catheter 20 and the waste fluid catheter 30. The slider 140 moves in a direction away from the housing 110 to pull the plunger 42 of the syringe 41 to generate a negative pressure to draw the dialysate out of a dialysate container. The dialysate from the dialysate container passes through the dialysate catheter 10, a four-way connector 50 and the syringe catheter 40 sequentially into the syringe 41.

Step S200: The dialysate is injected into a patient's abdominal cavity. The medical injection and suction device 100 opens the second valve 132 to make the injection catheter 20 conductive and closes the first valve 131 and the third valve 133 to close the dialysate catheter 10 and the waste fluid catheter 30. The slider 140 moves in a direction toward the housing 110, pushing the plunger 42 of the syringe 41 and thereby forcing the dialysate out of the syringe 41. The dialysate from the syringe 41 passes through the syringe catheter 40, the four-way connector 50, the infusion catheter 20, and the peritoneal catheter 24 sequentially into the abdominal cavity of the patient such that the dialysate comes into contact with the patient's peritoneum for a period of time to transfer intracorporeal wastes into the dialysate using the peritoneum as a semipermeable membrane for dialysis.

Step S300: The waste fluid containing the intracorporeal wastes is drained from the patient's abdominal cavity into the waste fluid container 33. The medical injection and suction device 100 opens the second valve 132 and the third valve 133 to make the infusion catheter 20 and the waste fluid catheter 30 conductive and closes the first valve 131 to close the dialysate catheter 10. The slider 140 is maintained at a position closest to the housing 110 to maintain the plunger 42 of the syringe 41 in a state of being pushed to the bottom. Thus, the pressure in the abdominal cavity forces the waste fluid containing the intracorporeal wastes to move toward the waste fluid container 33. The waste fluid from the abdominal cavity passes through the peritoneal catheter 24, the infusion catheter 20, the four-way connector 50 and the waste fluid catheter 30 sequentially into the waste fluid container 33.

In one or more embodiments, a user may input a procedure of injection and suction via the input interface (liquid crystal touch screen). Taking the above peritoneal dialysis for example, in step S100, the input may be opening the first valve 131, closing the second valve 132 and the third valve 133, and drawing a quantified dialysate at a predetermined rate (or for a predetermined amount of time). In step S200: the input may be opening the second valve 132, closing the first valve 131 and the third valve 133, and injecting the quantified dialysate at a predetermined rate for a residence time of 50 minutes. In step S300: the input may be opening the third valve 133, closing the first valve 131 and the second valve 132, draining the waste fluid for 10 minutes, and repeating the steps S100 to S300 a predetermined number of times. The control device 170 then automatically executes the input injection and suction procedure. In one or more embodiments, the user only needs to select a modular treatment program via the input interface (liquid crystal touch screen). The control device 170 then automatically executes all the input injection and suction procedure. Taking the above peritoneal dialysis for example, the user only needs to select “peritoneal dialysis” among the options of treatment programs and input the relevant background information of the patient, such as body weight. The control device 170 then calculates an appropriate injection amount of the dialysate, an injection time, a residence time, a waste fluid discharge time based on the body weight, and executes the appropriate injection and suction procedure.

To summarize, in the medical injection and suction device 100 of one or more embodiments disclosed herein, (1) the plurality of valves 131, 132, 133 are configured to close and open the plurality of catheters connected to the syringe, and the slider 140 is connected to the plunger 42 of the syringe 41 and pushes and pulls the plunger 42 of the syringe 41 to inject and suction the liquid. The amount of the injected infusion and the amount of the suctioned waste fluid are precisely quantified by using the displacement sensing device 190 to sense the displacement of the slider 140. The displacement sensing device 190 measures the displacement of the slider using the sensed resistance value of the resistance ruler 191 instead of using a conventional grating, thus improving the precision of quantification. (2) The medical injection and suction device 100 provides the slow but steady injection of a liquid in the syringe 41 into a patient's body at a specific rate by applying a steady force to the plunger 42 of the syringe 41 in order to overcome instability during injection resulting from pushing the fluid in the catheter traditionally by manually pushing the plunger 42 of the syringe 41 or by gravity. (3) The flow sensor 180 detects the flow velocity and the flow direction in the catheter 20 to prevent backflow or blockage in the catheter 20, eliminating the need for personal execution and monitoring by the medical personnel, which saves a lot of manpower. (4) The slider 140 is disposed on a lateral side of the housing 110 and does not extend through the wall of the upper side of the housing 110 to form a hole or slit in the wall 112 of the upper side, so that the leaked liquid does not pass through the hole or slit of the wall 112 of the upper side into the housing 110 and causes damage to the medical injection and suction device 100, thereby prolonging the product life. (5) The consumables used in conjunction with the medical injection and suction device 100 have a barcode on the package. The control device 170 can be started only after the barcode scanner 200 scans the barcode on the package. This ensures that, each time the medical injection and suction device 100 is used, new and clean consumables are used in conjunction with the device to avoid the repeated use of consumables, which leads to cross-infection.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

1. A medical injection and suction device comprising:
a housing that encloses a driving motor,
a syringe mount disposed on a wall of an upper side of the housing;
a plurality of valves disposed on the wall of the upper side of the housing and on a side of the syringe mount;
a slider that moves toward or away from the housing and is disposed on a lateral side of the housing;
a driving rod connected to the slider, wherein
the driving rod extends through a wall of the lateral side of the housing,
an end of the driving rod is connected to the driving motor, and
the driving motor drives the driving rod to move the slider, and
at least one slide rod, wherein
an end of the at least one slide rod is connected to the wall of the lateral side of the housing, and
the slider is disposed in a slidable manner on the slide rod.

2. The medical injection and suction device according to claim 1, wherein
the syringe mount fixes a syringe to the housing,
the plurality of valves closes and opens a plurality of catheters connected to the syringe, and
the slider is connected to a plunger of the syringe and pushes and pulls the plunger of the syringe to inject and suction liquids.

3. The medical injection and suction device according to claim 1, wherein the slider is not disposed on the upper side of the housing.

4. The medical injection and suction device according to claim 1, further comprising: a stop block that stops the slider from moving off the slide rod and is disposed at an end of the slide rod.

5. The medical injection and suction device according to claim 1, further comprising: a control device that is electrically connected to the driving motor and controls the driving of the driving motor to move the slider, wherein the control device is electrically connected to the plurality of valves and controls the closing and opening of the valves.

6. The medical injection and suction device according to claim 5, further comprising: a flow sensor that senses a flow velocity and a flow direction of a liquid in a catheter.

7. The medical injection and suction device according to claim 6,
wherein the control device is electrically connected to the flow sensor and receives a signal of the flow velocity and the flow direction sensed by the flow sensor,
and wherein the control device stops the movement of the slider and issues an alarm in response to the flow velocity being lower than a predetermined value, or the control device stops the movement of the slider and issues an alarm in response to the flow direction not being a predetermined direction.

8. The medical injection and suction device according to claim 5, further comprising: a displacement sensing device comprising a resistance ruler, wherein the displacement sensing device measures a displacement of the slider by reading a difference of resistance value of the resistance ruler.

9. The medical injection and suction device according to claim 8, wherein
the control device is electrically connected to the displacement sensing device and receives a signal of the displacement of the slider measured by the displacement sensing device, and
the control device stops the movement of the slider in response to the displacement of the slider measured by the displacement sensing device reaching a predetermined displacement to execute a predetermined injection amount or a predetermined suction amount.

10. The medical injection and suction device according to claim 9, wherein
the control device comprises a timing unit that calculates and measures time, and
the control device drives the driving motor for a predetermined amount of time for injection or a predetermined amount of time for suction to complete the predetermined displacement of the slider to execute a predetermined injection rate or a predetermined suction rate.

11. The medical injection and suction device according to claim 5, further comprising: a barcode scanner electrically connected to the control device, wherein the barcode scanner sends a start signal to the control device in response to the completion of scanning of a correct barcode, and wherein the control device cannot be started until the start signal is received.

12. The medical injection and suction device according to claim 1, further comprising: a control device, wherein the control device comprises an input interface for inputting a patient data, an output interface for outputting the patient data, and a memory that stores the patient data.

13. The medical injection and suction device according to claim 1, further comprising: a control device comprising a transceiving unit that uploads the patient data to an alarm in the control unit to a cloud terminal, a remote computer, or a mobile device.
